(19) United States
(12) Patent Application Publication
(10) Pub. No.: US 2026/0174320 A1
(43) Pub. Date: Jun. 25, 2026

(54) ENDOSCOPE REPROCESSOR, METHOD, AND CONTROL APPARATUS FOR FLUID SUPPLY CONTROL

(71) Applicant: OLYMPUS MEDICAL SYSTEMS CORP., Tokyo (JP)

(72) Inventor: Yukihiro MIKAWA, Tokyo (JP)

(73) Assignee: OLYMPUS MEDICAL SYSTEMS CORP., Tokyo (JP)

(21) Appl. No.: 19/532,446

(22) Filed: Feb. 6, 2026

Related U.S. Application Data
(63) Continuation of application No. PCT/JP2023/029253, filed on Aug. 10, 2023.

(51) Int. Cl. A61B 1/12; A61B 1/00

(52) U.S. Cl. CPC A61B 1/123; A61B 1/00128

(57) ABSTRACT

An endoscope reprocessor includes a first tube connectable to a first ferrule of an endoscope, a second tube connectable to a second ferrule of the endoscope, a fluid supplying assembly configured to supply a fluid to a conduit of the endoscope via the first and second tubes, a sensor configured to detect a pressure and/or a flow rate of the fluid in the conduit, and processing circuitry. The processing circuitry is configured to control the fluid supplying assembly in a first mode to supply the fluid at a first pressure to the first tube, acquire the pressure and/or the flow rate from the sensor, and based on the pressure and/or the flow rate, switch control of the fluid supplying assembly from the first mode to a second mode to supply the fluid at a second pressure higher than the first pressure to the first tube.

### CROSS REFERENCE TO RELATED APPLICATION

This application is a continuation of International Application No. PCT/JP2023/029253, filed on Aug. 10, 2023, the entire contents of which are incorporated herein by reference.

### FIELD

The present disclosure relates to an endoscope reprocessor, a method, and a control apparatus for fluid supply control in endoscope reprocessing.

### BACKGROUND

Endoscopes for use in medical fields are used to perform observation in a body by an insertion portion being inserted into a body and perform treatment with a treatment instrument, and thereafter, for reuse, need to be subjected to reprocessing such as cleaning and disinfecting. Endoscope reprocessors are used for performing automatic reprocessing safely and surely.

The endoscope reprocessors are configured such that connectors of respective tubes are connected respectively to a suction ferrule and a forceps port ferrule of an endoscope, and then a fluid is supplied to each of the tubes to be discharged from an opening of a distal end of an insertion portion, to thereby remove contaminants such as blood clots and mucus adhering to a conduit in the endoscope.

International Publication No. WO 2015-001843 discloses an endoscope cleaning apparatus configured to clean a forceps port ferrule using a liquid leaking from between the forceps port ferrule and a connector of a tube connected to the forceps port ferrule.

International Publication No. WO 2016-194456 discloses an endoscope reprocessor configured to perform flow control for adjusting a flow rate of a first fluid supplying unit for supplying a liquid as a fluid and a flow rate of a second fluid supplying unit for supplying a gas as a fluid.

### SUMMARY

According to aspects of the present disclosure, an endoscope reprocessor is provided, which includes a first tube, a second tube, a fluid supplying assembly, a sensor, and processing circuitry. The first tube is connectable to a first ferrule of an endoscope. The second tube is connectable to a second ferrule of the endoscope. The fluid supplying assembly is configured to supply a fluid to a conduit of the endoscope via the first and second tubes. The sensor is configured to detect at least one of a pressure or a flow rate of the fluid in the conduit. The processing circuitry is configured to control the fluid supplying assembly in a first mode to supply the fluid at a first pressure to the first tube. The processing circuitry is further configured to acquire at least one of the pressure or the flow rate from the sensor. The processing circuitry is further configured to switch, based on at least one of the pressure or the flow rate, control of the fluid supplying assembly from the first mode to a second mode to supply the fluid at a second pressure higher than the first pressure to the first tube.

According to aspects of the present disclosure, further provided is a method implementable by processing circuitry operatively coupled to an endoscope reprocessor. The method includes controlling a fluid supplying assembly in a first mode to supply a fluid at a first pressure to a first tube. The method further includes acquiring at least one of a pressure or a flow rate of the fluid in a conduit of an endoscope from a sensor. The method further includes switching, based on at least one of the pressure or the flow rate, control of the fluid supplying assembly from the first mode to a second mode to supply the fluid at a second pressure higher than the first pressure to the first tube. The endoscope reprocessor includes the first tube connectable to a first ferrule of the endoscope. The endoscope reprocessor further includes a second tube connectable to a second ferrule of the endoscope. The endoscope reprocessor further includes the fluid supplying assembly configured to supply the fluid to the conduit of the endoscope via the first and second tubes. The endoscope reprocessor further includes the sensor configured to detect at least one of the pressure or the flow rate of the fluid in the conduit.

According to aspects of the present disclosure, further provided is a control apparatus that includes a connection interface and processing circuitry. The connection interface is connectable to a fluid supplying assembly and a sensor. The fluid supplying assembly is configured to supply a fluid to a conduit of an endoscope via a first tube and a second tube. The first tube is connectable to a first ferrule of the endoscope. The second tube is connectable to a second ferrule of the endoscope. The sensor is configured to detect at least one of a pressure or a flow rate of the fluid in the conduit. The processing circuitry is configured to control the fluid supplying assembly in a first mode to supply the fluid at a first pressure to the first tube. The processing circuitry is further configured to acquire at least one of the pressure or the flow rate from the sensor. The processing circuitry is further configured to switch, based on at least one of the pressure or the flow rate, control of the fluid supplying assembly from the first mode to a second mode to supply the fluid at a second pressure higher than the first pressure to the first tube.

### BRIEF DESCRIPTION OF DRAWINGS

FIG. 1 is a perspective view of an endoscope reprocessor in an embodiment.

FIG. 2 is a configuration view of a main part of the endoscope reprocessor in the embodiment.

FIG. 3 is a cross-sectional view of a connecting part between a connector of a tube of the endoscope reprocessor in the embodiment and a forceps port ferrule.

FIG. 4 is a cross-sectional view of a conduit merging portion of an endoscope connected to the endoscope reprocessor in the embodiment.

FIG. 5 is a flowchart of an operation method for the endoscope reprocessor in the embodiment.

FIG. 6 is a flowchart of the operation method for the endoscope reprocessor in the embodiment.

### DESCRIPTION OF EMBODIMENTS

Hereinafter, an endoscope reprocessor 1 in an embodiment of the present disclosure will be described with reference to drawings. Hereinafter, the endoscope reprocessor 1 is referred to as a reprocessor 1.

Note that the drawings based on the embodiment are schematic. The relationship between thicknesses and widths of respective parts, a ratio of thicknesses, and the like of the respective parts are different from the actual ones. The respective drawings include parts in which the relationships and ratios among the dimensions are different.

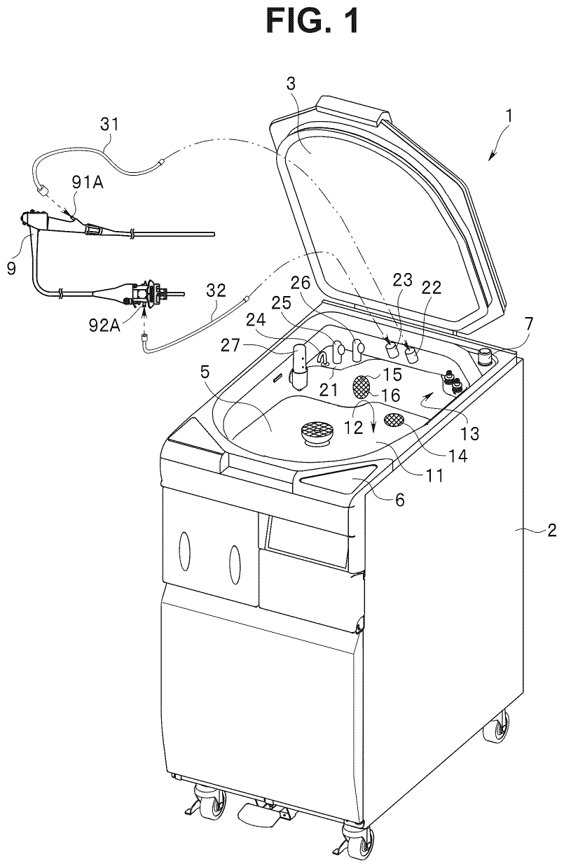

As shown in FIG. 1, the reprocessor 1 includes a main body 2, and an openable/closable top cover 3. FIG. 1 shows a state where the top cover 3 of the reprocessor 1 is open.

The reprocessor 1 is an apparatus configured to perform reprocessing (reproducing processing) of an endoscope 9 or endoscope accessories. The reprocessing may be any of cleaning for removing contaminants such as organic matters, disinfection for disabling certain microorganisms, sterilization for eliminating or killing all the microorganisms, or a combination of these.

The main body 2 includes, at an upper portion thereof, a processing tank 5 in which the endoscope 9 is subjected to processing such as cleaning and disinfecting, an operation panel 6, and a water supplying hose connecting port 7.

The processing tank 5 stores a liquid such as a cleaning solution, water, an alcohol-based disinfectant solution, or a sterile solution. The processing tank 5 includes an endoscope arranging portion 11, and a terrace 21.

The endoscope arranging portion 11 includes a bottom surface 12 and a side surface 13, and is configured such that the endoscope 9 can be arranged therein. Further, the endoscope arranging portion 11 stores the liquid. The endoscope arranging portion 11 includes, on the bottom surface 12, a discharge port 14 from which the stored liquid is discharged. The endoscope arranging portion 11 includes, on the side surface 13, a circulation port 16 including a mesh filter 15. The circulation port 16 is in communication with a liquid pump 51 to be described later. The circulation port 16 may be provided on the bottom surface 12.

The terrace 21 is adjacent to the endoscope arranging portion 11, and located at a position higher than the position of the endoscope arranging portion 11. The terrace 21 includes a water supplying port 22, a gas feeding port 23, a cleaning solution nozzle 24, a disinfectant solution nozzle 25, a water supplying nozzle 26, and a liquid level sensor 27.

The water supplying port 22 is a port for connecting a first tube 31. The gas feeding port 23 is a port for connecting a second tube 32. The number of the ports included in the reprocessor 1 is not limited to two.

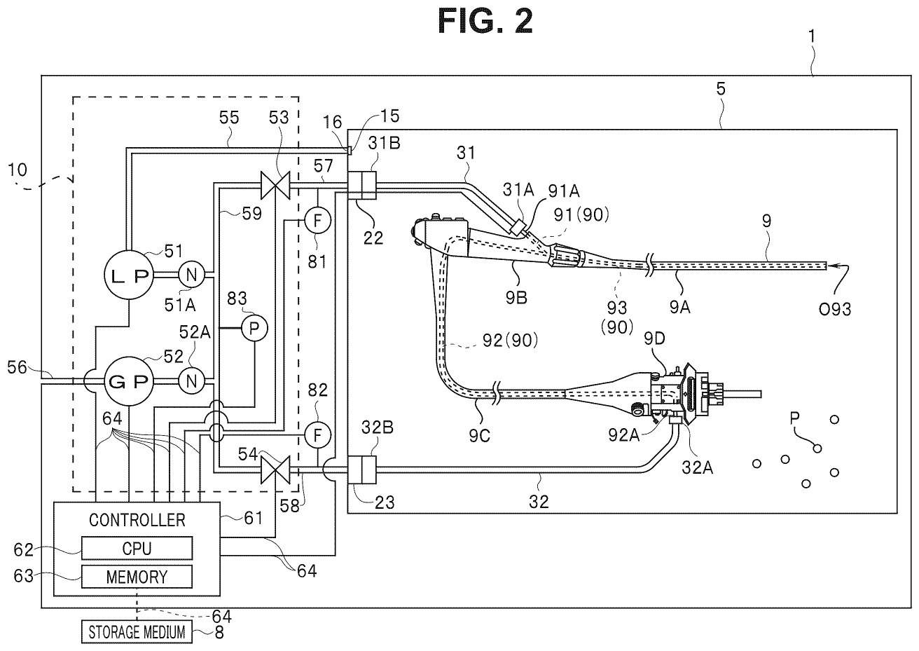

The cleaning solution nozzle 24 is configured to supply a cleaning solution to the processing tank 5. The disinfectant solution nozzle 25 is configured to supply a disinfectant solution to the processing tank 5. The water supplying nozzle 26 is configured to supply water taken in from the water supplying hose connecting port 7 to the processing tank 5, and supply the liquid in the processing tank 5, which has been taken in from the circulation port 16 having the mesh filter 15, again to the processing tank 5, to cause the liquid to circulate. The mesh filter 15 is configured to filter contaminants P (see FIG. 2) in the liquid. The liquid level sensor 27 is configured to detect a liquid level of the liquid stored in the processing tank 5.

The operation panel 6 is arranged on an upper front portion of the main body 2. The operation panel 6 includes various operation buttons and a display panel that are not shown. A user gives various instructions to the reprocessor 1 through the use of the operation panel 6.

The water supplying hose connecting port 7 is provided at an upper rear portion of the main body of the reprocessor. A water supplying hose, which is connected to a water faucet not shown, is connected to the water supplying hose connecting port 7, to thereby supply water to the reprocessor 1 via the water supplying nozzle 26.

The top cover 3 is provided at an upper portion of the main body 2 so as to be openable/closable. In the reprocessor 1, the top cover 3 is brought into an open state, the endoscope 9 is arranged in the endoscope arranging portion 11, and the endoscope 9 and the reprocessor 1 can be connected to each other by using the first tube 31 and the second tube 32. After setting the endoscope 9, the top cover 3 is closed, thereby bringing the reprocessor 1 into a reprocessing enabled state.

FIG. 2 shows a state where the endoscope 9 to which the first tube 31 and the second tube 32 are connected is housed in the reprocessor 1. Note that FIG. 2 shows only the main configuration in the present disclosure. The reprocessor 1 may have a configuration different from the configuration shown in FIG. 2, as long as it has functions which are the same as those in the configuration shown in FIG. 2.

The endoscope 9 includes an insertion portion 9A configured to be inserted into a body, an operation portion 9B, a universal cord 9C, and an endoscope connector 9D. The endoscope 9 includes, inside thereof, a conduit 90. The conduit 90 includes a first conduit 91, a second conduit 92, and a third conduit 93. The first conduit 91 includes, at one end thereof, a forceps port ferrule 91A which is a first ferrule, and the other end of the first conduit 91 is merged with the second conduit 92 at a merging portion. The second conduit 92 includes, at one end thereof, a gas feeding ferrule 92A which is a second ferrule, and the other end of the second conduit 92 is merged with the first conduit 91 at the merging portion. In other words, the first conduit 91 is in communication with the forceps port ferrule 91A and the second conduit 92 is in communication with the gas feeding ferrule 92A.

The third conduit 93 in which the first conduit 91 and the second conduit 92 are merged, passes through the insertion portion 9A, and includes an opening O93 at the distal end of the insertion portion 9A. The first conduit 91 and the third conduit 93 constitute a forceps channel. A treatment instrument such as a forceps, which is inserted from the forceps port ferrule 91A, passes through the first conduit 91 and the third conduit 93 and the distal end of the treatment instrument is protruded from the opening O93. A fluid fed from the gas feeding ferrule 92A is emitted from the opening O93, via the second conduit 92 and the third conduit 93. Note that the gas feeding ferrule 92A is not dedicated to gas feeding during the use of the endoscope 9, but is used also for suctioning or water feeding.

The first tube 31 is configured such that a first connector 31A provided at the one end is connected to the forceps port ferrule 91A which serves as the first ferrule of the endoscope 9, and a connector 31B provided at the other end is connected to the water supplying port 22 of the reprocessor 1. The water supplying port 22 is in communication with the forceps port ferrule 91A of the endoscope 9, via the first tube 31.

The second tube 32 is configured such that a second connector 32A provided at the one end is connected to the gas feeding ferrule 92A which serves as the second ferrule of the endoscope 9, and a connector 32B provided at the other end is connected to the gas feeding port 23 of the reprocessor 1. The gas feeding port 23 is in communication with the gas feeding ferrule 92A of the endoscope 9, via the second tube 32.

The reprocessor 1 includes a liquid pump 51, a gas pump 52, a first solenoid valve 53, a second solenoid valve 54, and a controller 61.

The liquid pump 51 takes the liquid such as the cleaning solution in the processing tank 5 into the conduit 55 through the circulation port 16 having the mesh filter 15, pressurizes the taken-in liquid, and feeds the pressurized liquid to a branch conduit 59. Although not shown, a part of the liquid taken in from the circulation port 16 is fed to the processing tank 5 by another pump. The mesh filter 15 is configured to filter the contaminants P streamed down from the endoscope 9 and floating in the liquid in the processing tank 5.

The liquid pump 51 is connected to the first solenoid valve 53 via the branch conduit 59. The first solenoid valve 53 is connected to the water supplying port 22 via the conduit 57.

The gas pump 52 takes in a gas via a conduit 56, pressurizes the taken-in gas, and feeds the pressurized gas to the branch conduit 59. The gas is air, for example. The gas pump 52 is connected to the second solenoid valve 54 via the branch conduit 59. The second solenoid valve 54 is connected to the gas feeding port 23 via a conduit 58.

A check valve 51A is disposed in a discharge conduit of the liquid pump 51. A check valve 52A is disposed in a discharge conduit of the gas pump 52. The check valves 51A and 52A are not essential constituent elements.

The branch conduit 59 is provided with a pressure sensor 83 for detecting a pressure of a fluid. The conduit 57 is provided with a flow rate sensor 81, and the conduit 58 is provided with a flow rate sensor 82.

A controller 61 may be implemented by processing circuitry including one or more processors. For instance, the controller 61 includes processing circuitry including a CPU 62, which is a central processing unit of a computer, and a memory 63 including a ROM, a RAM, and/or the like. Functions of the controller 61 may be achieved by the CPU 62 reading a program from the memory 63 and executing the program. The program for causing a computer to perform reprocessing, which is stored in the memory 63, may be stored in a non-transitory computer readable storage medium 8 and transferred to the memory 63. The memory 63 also stores a plurality of thresholds corresponding to a model of the endoscope 9 to be described later.

The controller 61 is electrically connected, via connection interfaces 64, to the liquid pump 51, the gas pump 52, the first solenoid valve 53, the second solenoid valve 54, the flow rate sensors 82, and the pressure sensor 83. Each connection interface 64 may include an electrical line and a connector through which control signals or detection signals are transmitted between the controller 61 and a respective one of the connected elements. Furthermore, the controller 61 may be implemented as a separate control apparatus operatively coupled to the reprocessor 1 in a wired or wireless manner. In this case, the controller 61 and the reprocessor 1 may form an endoscope reprocessing system.

When supplying the liquid, the controller 61 activates the liquid pump 51 and stops the gas pump 52. When supplying the gas, the controller 61 stops the liquid pump 51 and activates the gas pump 52.

After the controller 61 activates the liquid pump 51 and then the gas pump 52, a gas-liquid mixed flow is supplied to the conduit 90. An operation of supplying the liquid and then feeding air from the gas pump 52 is repeated, and thereby the fluid to be supplied becomes the gas-liquid mixed flow in which the liquid and the gas are mixed. The gas-liquid mixed flow includes all of a state where air bubbles exist in the liquid, a state where droplets exist in the gas, and a state where a liquid mass and a gaseous mass exist together.

In addition, the controller 61 controls the opening/closing state of the first solenoid valve 53, to thereby cause the fluid at a predetermined flow rate (a predetermined pressure) to be supplied to the first conduit 91 via the first tube 31. The controller 61 controls the opening/closing state of the second solenoid valve 54, to thereby cause the fluid at the predetermined flow rate to be supplied to the second conduit 92 via the second tube 32.

The liquid pump 51, the gas pump 52, the first solenoid valve 53, the second solenoid valve 54, the pressure sensor 83 and the like constitute a fluid supplying unit (hereinafter may be referred to as a fluid supplying assembly) 10. The fluid supplying unit 10 is configured to supply a fluid to each of the first tube 31 and the second tube 32. The controller 61 is configured to control a timing at which the fluid supplying unit 10 supplies the fluid to the conduit 90.

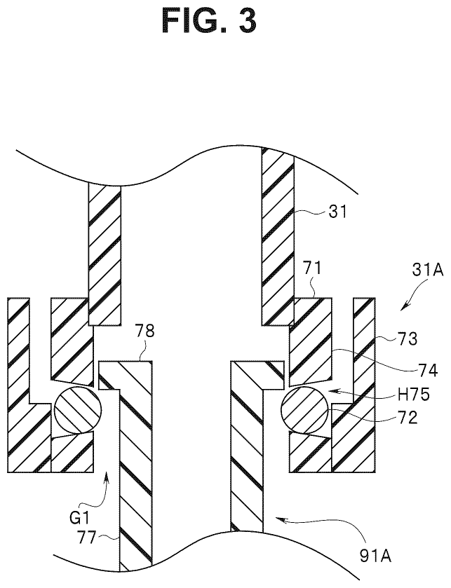

FIG. 3 is a cross-sectional view of a connecting portion between the first connector 31A of the first tube 31 and the forceps port ferrule 91A of the endoscope 9. Note that, since a connecting portion between the second connector 32A of the second tube 32 and the gas feeding ferrule 92A of the endoscope 9 has the same configuration as that of the connecting portion between the first connector 31A and the forceps port ferrule 91A, description thereof will be omitted.

The first connector 31A includes a connector main body 71 provided at a distal end of the first tube 31, a plurality of spherical bodies 72, and a connector cover 73 provided on an outer circumferential portion of the connector main body 71.

The connector main body 71 is formed of plastic, or the like. The connector main body 71 has a cylindrical shape, and includes, at a circumferential lateral portion 74 thereof, a plurality of circular holes H75. The holes H75 include, for example, four holes provided at equal intervals along a circumferential direction in the circumferential lateral portion 74 of the connector main body 71. Each of the holes H75 has a diameter reduced from the outer surface toward the inner surface of the connector main body 71, and the circumferential lateral portion 74 has a tapered cross section in the thickness direction.

The plurality of spherical bodies 72 are formed of metal, or the like. In order to inhibit each of the plurality of spherical bodies 72 from falling off from the inner circumferential surface of the circumferential lateral portion 74, each of the plurality of spherical bodies 72 has a diameter larger than the diameter of each of the holes H75 in the inner circumferential surface of the circumferential lateral portion 74, and is arranged such that a part thereof enters each of the holes H75.

The connector cover 73 is formed of plastic, or the like. The connector cover 73 is arranged at an outside of the connector main body 71 provided with the plurality of spherical bodies 72.

The forceps port ferrule 91A is formed of metal, a resin, or the like. The forceps port ferrule 91A includes a body portion 77 formed in a cylindrical shape. The forceps port ferrule 91A includes, at the distal end thereof, an outward flange 78.

The first connector 31A of the first tube 31 is detachably connected to the forceps port ferrule 91A. In the state where the first connector 31A is attached to the forceps port ferrule 91A, the plurality of (four in the present embodiment) spherical bodies 72 of the first connector 31A lock the outward flange 78 of the forceps port ferrule 91A to inhibit the outward flange 78 from falling off. A circumferential gap G1 is formed between the body portion 77 of the forceps port ferrule 91A and the connector main body 71.

The fluid fed from the first tube 31 is introduced into the inside of the body portion 77 of the forceps port ferrule 91A, and flows out from the gap G1, via the part between the connector main body 71, in which the spherical bodies 72 are disposed in the holes H75, and the forceps port ferrule 91A. The fluid flowed out from the gap G1 in the connecting region between the first connector 31A and the forceps port ferrule 91A cleans the outer circumferential surface of the forceps port ferrule 91A.

Note that the shape of the first connector 31A of the first tube 31 is not limited to the above-described one, and for example, the connector disclosed in the International Publication No. WO 2015-001843, which has already described above, can be applied.

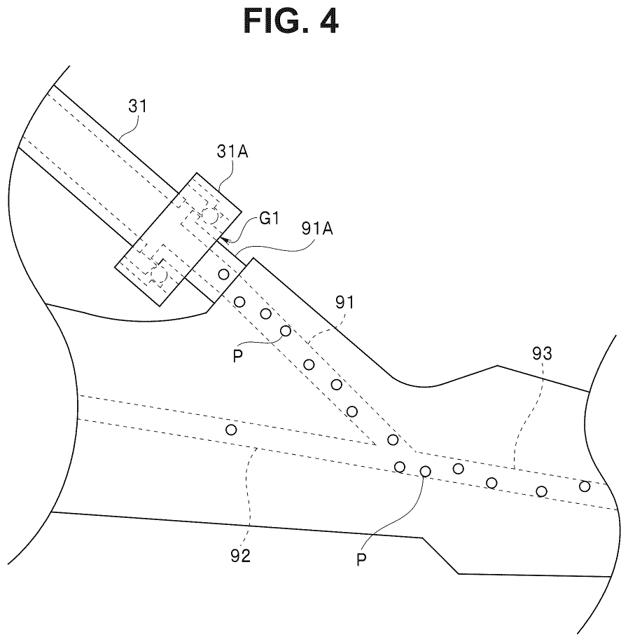

As shown in FIG. 4, there is a possibility that the contaminants P adhere to the conduit 90 of the endoscope 9 after use, in particular, the first conduit 91 and the third conduit 93 that are extraction paths of the treatment instrument inserted into a body.

As already described above, the gap G1 is present between the forceps port ferrule 91A and the first connector 31A of the first tube 31 connected to the forceps port ferrule 91A. Accordingly, in the reprocessing, for example, if the fluid supplied to the second conduit 92 flows reversely through the first conduit 91 via the merging portion, the contaminants P swept away by the fluid may possibly be caught in the gap G1.

As described later, the controller 61 controls the timing and the pressure at which the fluid supplying unit 10 supplies the fluid to the conduit 90. Specifically, the controller 61 compares the pressure of the fluid which has been detected by the pressure sensor 83 with a plurality of predetermined thresholds, and based on a result of the comparison, switches the control of the fluid supplying unit 10.

In other words, as described later, the controller 61 switches the control of the fluid supplying unit 10 from a first mode to a second mode, based on the result of the comparison. In the second mode, the fluid at a second pressure P2 higher than a first pressure P1 in the first mode is supplied to the first tube 31.

With the above-described control, even if the contaminants P in the first conduit 91 are clogged in the gap G1, the clogged contaminants can be removed. As a result, the endoscope reprocessor 1 can perform efficient reprocessing.

### <Operation Method for Endoscope Reprocessor>

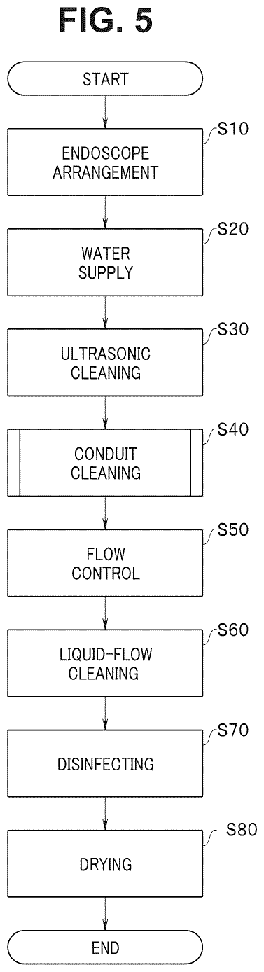

Description will be made on an example of the operation method for the reprocessor 1 according to the flowchart shown in FIG. 5.

### <Step S10> Endoscope Arranging Step

The user opens the top cover 3 of the reprocessor 1 and sets the endoscope 9. Specifically, the user connects the second connector 32A of the second tube 32 to the gas feeding ferrule 92A of the endoscope 9, and connects the connector 32B to the gas feeding port 23.

Then, the user connects the first connector 31A of the first tube 31 to the forceps port ferrule 91A of the endoscope 9, and connects the connector 31B to the water supplying port 22.

After connecting the reprocessor 1 and the endoscope 9, the user arranges the endoscope 9 in the endoscope arranging portion 11, and closes the top cover 3.

When the user gives an instruction for starting predetermined reprocessing such as cleaning and disinfecting from the operation panel 6, the CPU 62 reads the predetermined program from the memory 63 to start the processing in the program. The program for the endoscope reprocessor, which is stored in the non-transitory computer readable storage medium 8, for example, is transferred, in advance, to the memory 63.

### <Step S20> Water Supplying Step

Based on the control signal from the CPU 62, water is supplied from the water supplying nozzle 26 to the processing tank 5. When the liquid level of the processing tank 5 detected by the liquid level sensor 27 reaches a predetermined level, the water supply automatically stops.

<step S30> Ultrasonic Cleaning Step

When a transducer, not shown, disposed on the bottom surface of the processing tank 5 is activated, ultrasonic waves are applied to the water stored in the processing tank 5. With the ultrasonic cleaning, the contaminants P on the outer surface of the endoscope 9 is cleaned.

### <Step S40> Conduit Cleaning Step

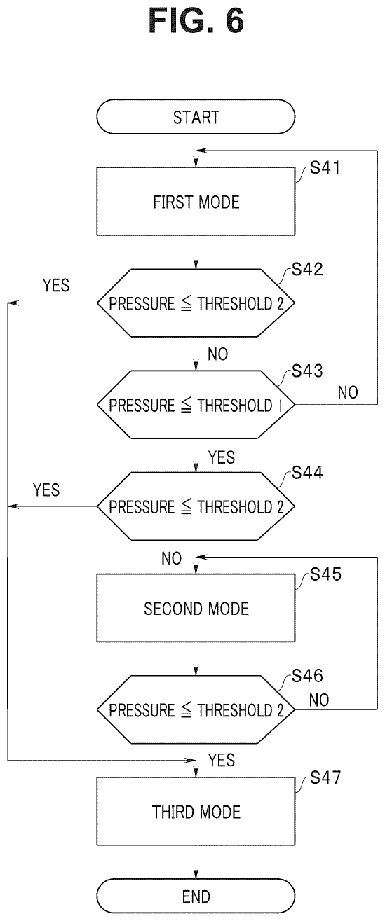

Details of the conduit cleaning step are shown in the flowchart in FIG. 6.

### <Step S41> First Mode

The controller 61 controls the fluid supplying unit 10 in the first mode. In the first mode, the controller 61 brings the first solenoid valve 53 into the “open” state, and controls the fluid supplying unit 10 so as to supply the water at the predetermined flow rate to the first conduit 91, via the first tube 31, the first connector 31A, and the forceps port ferrule 91A.

The flow rate control is performed by controlling the opening level of the first solenoid valve 53 or controlling the driving power of the pump 51.

### <Step S42> Pressure ≤Threshold 2

The first mode is a mode for checking whether the contaminants P adhere to the conduit 90 and removing the contaminants P in the conduit 90. When the pressure of the pressure sensor 83 is equal to or lower than a second threshold T2 (YES), the controller 61 determines that the removing process of the contaminants P in the conduit 90 is unnecessary, to move on to Step S47.

Note that the plurality of thresholds such as the second threshold T2 and a first threshold T1 to be described later are acquired in advance based on the model of the endoscope 9 and stored in the memory 63. The data of the model of the endoscope 9 to be reprocessed may be inputted by the user through the operation panel 6, or if an RFID tag is attached to the endoscope 9, the reprocessor 1 may automatically acquire the data.

In a case where the contaminants in an amount exceeding a predetermined amount adhere to the conduit 90, the pressure becomes greater than the first threshold T1. Although the contaminants in an amount exceeding the predetermined amount does not adhere to the conduit 90, in a case where the contaminants P are clogged in the gap G1, the pressure becomes greater than the second threshold T2. In other words, the first threshold T1 is greater than the second threshold T2. When the pressure is equal to or lower than the second threshold T2, the contaminants in an amount exceeding the predetermined amount does not adhere to the conduit 90, and the contaminants P are not clogged in the gap G1.

### <Step S43> Pressure ≤Threshold 1

When the pressure of the pressure sensor 83 is greater than the first threshold T1 (S43: NO), the controller 61 moves on to Step S41 in order to remove the contaminants P in the conduit 90, and continues the control in the first mode.

### <Step S44> Pressure≤Threshold 2

When the pressure of the pressure sensor 83 is equal to or lower than the first threshold T1 and equal to or lower than the second threshold T2 (S44: YES), it indicates that the contaminants P in the conduit 90 are removed and the contaminants P are not clogged in the gap G1, and the controller 61 moves on to the processing in Step S47.

### <Step S45> Second Mode

When the pressure of the pressure sensor 83 is equal to or lower than the first threshold T1 and greater than the second threshold T2 (S44: NO), the controller 61 controls the fluid supplying unit 10 in the second mode. In other words, the thresholds include the first threshold T1 for detecting the contaminants P in the conduit 90, and the second threshold T2 for detecting the clogging of the gap G1 due to the contaminants P.

In the second mode, the water at the second pressure P2 is supplied to the first conduit 91. The second pressure P2 is greater than the first pressure P1. The second mode is a mode for removing the clogging of the gap G1 due to the contaminants P.

The second pressure P2 is preferably greater than 1.5 times the first pressure P1, and in particular, is preferably greater than twice the first pressure P1. If the second pressure P2 is greater than the above-described pressure, the clogging of the gap G1 due to the contaminants P can be removed effectively.

When the fluid supplying unit 10 includes a plurality of pumps 51, the controller 61 may control the fluid supplying unit 10 to supply the fluid using the larger number of pumps 51 in the second mode than the number of pumps 51 in the first mode.

### <Step S46> Pressure≤Threshold 2

The controller 61 continues the control in the second mode (Step S45) until the pressure of the pressure sensor 83 becomes equal to or lower than the second threshold T2, that is, until the clogging of the gap G1 due to the contaminants P can be removed.

After a first predetermined time period (for example, 10 seconds) has elapsed from the switching of the control of the fluid supplying unit 10 to the second mode, the controller 61 may move on to Step S47. Hereinafter, the first predetermined time period may simply be referred to as a predetermined time period.

After a second predetermined time period (for example, 5 seconds) has elapsed from the switching of the control of the fluid supplying unit 10 to the second mode, the controller 61 may switch the control again to the control to the first mode. In other words, the control in the first mode in which the low pressure liquid is fed and the control in the second mode in which the high pressure liquid is fed may be performed repeatedly. The repeat count is more than twice but less than five times, for example. Hereinafter, the second predetermined time period may be referred to as a first particular time period.

When the controller 61 has performed the control of the fluid supplying unit 10 in the second mode for longer than a third predetermined time period (for example, 10 seconds), the controller 61 may generate an alarm signal. The alarm signal is transmitted to the user by displaying it as an image or characters on the operation panel 6, or generating a buzzer sound, for example. In other words, if the clogging is not eliminated even if the liquid feeding is performed at the high pressure for the third predetermined time period, the user performs cleaning with a brush, for example. Hereinafter, the third predetermined time period may be referred to as a second particular time period.

<step S47> Third Mode

The controller 61 switches the control of the fluid supplying unit 10 to a third mode in which the fluid is supplied to the first tube 31 and the second tube 32. A third pressure P3 of the fluid in the third mode is lower than the first pressure P1. In the third mode, the contaminants P in the second conduit 92 are also removed.

With the control in the third mode, the contaminants removing processing in the conduit 90 is almost completed.

Step S40 may include a time period during which the first solenoid valve 53 is brought into a “closed” state, so that only the liquid is fed to the second tube 32. In addition, Step S40 may include a time period during which the gas pump 52 is activated, and the gas-liquid mixed flow is supplied to the conduit 90.

The controller 61 can also control the fluid supplying unit 10 by comparing the flow rate detected by the flow rate sensor 81 disposed in the conduit 57 and the flow rate detected by the flow rate sensor 82 disposed in the conduit 58 with the plurality of thresholds.

When the flow rate is equal to or greater than a first threshold and lower than a second threshold, the controller 61 performs switching to the second mode, with a second flow rate greater than the first flow rate in the first mode, to control the fluid supplying unit 10. The second flow rate is preferably greater than 1.5 times the first flow rate, and in particular, is preferably greater than twice the first flow rate. If the second flow rate is greater than the above-described flow rate, the clogging of the gap G1 due to the contaminants P can be removed effectively.

<step S50> Flow Control Step

For example, the flow control step disclosed in the already described International Publication No. WO2016-194456 is performed. In other words, the flow rate at which the liquid is supplied as the fluid, the flow rate at which the gas is supplied as the fluid, the first solenoid valve 53, and the second solenoid valve 54 are adjusted, to thereby cause the liquid, the gas, or the gas-liquid mixed flow to be supplied in a predetermined order to the first conduit 91 or the second conduit 92.

<Step S60> Liquid-flow Cleaning Step

The cleaning solution in a cleaning solution tank is poured from the cleaning solution nozzle 24 into the processing tank 5 in which the water is stored. The cleaning solution diluted with the water is discharged from the circulation port 16 by the liquid pump 51, and supplied again to the processing tank 5 and the conduit 90.

<Step S70> Disinfecting Step

After the diluted cleaning solution is discharged from the processing tank 5, the disinfectant solution in a disinfectant solution tank is poured from the disinfectant solution nozzle 25 into the processing tank 5. The disinfectant solution is sucked from the circulation port 16 by the liquid pump 51, and supplied again to the processing tank 5 and the conduit 90.

### <Step S80> Drying Step

After the disinfectant solution is discharged from the processing tank 5, air is fed into the conduit 90 by the gas pump 52, and drying processing for removing the water in the conduit is performed. A liquid for drying such as alcohol, not shown, may be fed to the conduit 90.

Then, the reprocessing of the endoscope 9 arranged in the processing tank 5 is completed. Note that the reprocessing is not limited to the above-described processing. For example, between the respective steps, a rinsing step with water and a drying step may be performed. In addition, the flow control step S50 may be omitted.

The fluid to be used for the conduit cleaning is not limited to the liquid. The gas-liquid mixed flow may be used. Furthermore, the liquid and the gas-liquid mixed flow may be used in combination. For example, the liquid may be used in the first mode, and the liquid and the gas-liquid mixed flow may be used in combination in the second mode.

The liquid to be used for the conduit cleaning is not limited to water. A cleaning solution, a disinfectant solution, or the like may be used. Furthermore, a warmed liquid may be used as the liquid.

There is a case where the contaminants P in a dry state can be removed more easily than the contaminants P containing moisture. In this case, before switching from the first mode to the second mode, a gas may be supplied for a predetermined time period to perform drying processing for removing the moisture of the contaminants P. Hereinafter, the predetermined time period in this case may be referred to as a specific time period.

In addition, a third threshold (hereinafter may be referred to as a particular threshold) T3 for detecting a connection between the first connector 31A and the forceps port ferrule 91A may be stored in the memory 63. The controller 61 may generate an alarm signal, when the pressure is equal to or lower than the third threshold T3 in Step S42.

In addition, in some cases, the endoscope 9 is of a model in which there is no possibility that the contaminants P adhere to the conduit 90 after use. The controller 61 may omit the control (S41-S46) started from the first mode according to the acquired data of the model of the endoscope 9, and start the control in the third mode.

As described above, an operation method for an endoscope reprocessor includes: controlling a fluid supplying unit in a first mode in which a fluid at a first pressure is supplied to a first tube; acquiring a pressure or a flow rate from a sensor; comparing the pressure or the flow rate with a plurality of thresholds corresponding to a model of an endoscope acquired in advance; and based on a result of the comparison, switching the control of the fluid supplying unit from the first mode to a second mode in which the fluid at a second pressure higher than the first pressure is supplied to the first tube, or to a third mode in which the fluid is supplied to the first tube and a second tube.

An operation program for an endoscope reprocessor causes a computer to perform control for controlling a fluid supplying unit in a first mode in which a fluid at a first pressure is supplied to a first tube; acquiring a pressure or a flow rate from a sensor; comparing the pressure or the flow rate with a plurality of thresholds corresponding to a model of an endoscope acquired in advance; and based on a result of the comparison, switching the control of the fluid supplying unit from the first mode to a second mode in which the fluid at a second pressure higher than the first pressure is supplied to the first tube, or to a third mode in which the fluid is supplied to the first tube and a second tube.

The endoscope 9 in the embodiment is a flexible endoscope for a medical use, but the endoscope in the present disclosure may be a rigid endoscope and may be for an industrial use. The endoscope 9 may be configured such that a monitor (not shown) may be directly connected to the operation portion 9B.

The present disclosure is not limited to the above-described embodiment, or the like, but various changes, modifications, etc., are possible without changing the gist of the present disclosure.

### (General Interpretation Notes)

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another.

The term “processing circuitry,” as used herein, refers to any hardware or combination of hardware and software configured to execute the operations described. The term “processing circuitry” is a broad structural term that encompasses, without limitation, general-purpose processors (e.g., CPUs, GPUs), application-specific integrated circuits (ASICs), field-programmable gate arrays (FPGAs), programmable logic devices (PLDs), and discrete logic circuits. In addition to logic or execution units, the processing circuitry may explicitly include or be integrally coupled to memory (e.g., registers, cache, RAM, or other storage media) that stores data, software, or instructions contributing to the processing operations. Accordingly, the processing circuitry may be implemented as a specialized hardware circuit having fixed logic, a programmable circuit executing instructions stored in an internal or external memory, or any combination thereof. Furthermore, like the “processor” described above, the processing circuitry may be distributed across multiple devices or locations (e.g., cloud computing) or consolidated within a single device. The term “processing circuitry” implies a concrete structure and is not intended to be construed as a purely functional “means” lacking structural support.

The term “non-transitory computer-readable (storage) medium” refers to any tangible device or medium capable of storing code or data for access by a computer or processing circuitry. This term encompasses a single storage medium or a group of multiple storage media, which may be locally or remotely distributed (e.g., across a network, in a cloud computing environment, or within a distributed ledger system) and may collectively store information in a coordinated or distributed manner. Examples of such media include, but are not limited to, non-volatile media (e.g., optical disks, magnetic disks, flash memory, ROM) and volatile media (e.g., dynamic memory, RAM, registers, buffers, and caches). Importantly, the term “non-transitory” is intended to exclude only transitory propagating signals per se (e.g., carrier waves, electromagnetic waves, or digital signals in transit through a transmission medium) and does not exclude statutory subject matter such as volatile memory where data is stored temporarily.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:
Clause 1: An endoscope reprocessor comprising:
   a first tube including a first connector configured to be connected to a first ferrule of an endoscope;
   a second tube including a second connector configured to be connected to a second ferrule of the endoscope;
   a fluid supplying unit configured to supply a fluid to a conduit of the endoscope via the first tube and the second tube;
   a controller configured to control the fluid supplying unit; and
   a sensor configured to detect a pressure or a flow rate of the fluid in the conduit, wherein the controller is configured to:
   control the fluid supplying unit in a first mode in which the fluid at a first pressure is supplied to the first tube;
   acquire the pressure or the flow rate from the sensor; and
   switch, based on the pressure or the flow rate, the control of the fluid supplying unit from the first mode to a second mode in which the fluid at a second pressure higher than the first pressure is supplied to the first tube.
Clause 2: The endoscope reprocessor according to clause 1, wherein
   the first connector is configured such that a part of the fluid supplied from the first tube leaks out from a gap between the first connector and the first ferrule to an outer circumferential surface of the first ferrule, and
   the controller compares the pressure or the flow rate with a plurality of thresholds corresponding to a model of the endoscope acquired in advance, and
   switches, based on a result of the comparison, the control of the fluid supplying unit from the first mode to the second mode, or to a third mode in which the fluid is supplied to the first tube and the second tube.
Clause 3: The endoscope reprocessor according to clause 2, wherein
   the plurality of thresholds include a first threshold for detecting contaminants adhering to the conduit and a second threshold for detecting a clogging of the gap due to the contaminants, and
   when the result of the comparison indicates that the pressure is equal to or lower than the first threshold and greater than the second threshold, the controller switches the control of the fluid supplying unit to the second mode.
Clause 4: The endoscope reprocessor according to clause 3,
   wherein the controller switches the control of the fluid supplying unit to the third mode, after a first predetermined time period has elapsed from the switching of the control of the fluid supplying unit to the second mode, or when the pressure becomes equal to or lower than the second threshold.
Clause 5: The endoscope reprocessor according to clause 4,
   wherein, according to the model of the endoscope acquired in advance, the controller switches the control of the fluid supplying unit to the control started from the first mode, or to the control started from the third mode.
Clause 6: The endoscope reprocessor according to clause 2,
   wherein the controller switches the control of the fluid supplying unit to the first mode again, after a second predetermined time period has elapsed from the switching to the second mode.
Clause 7: The endoscope reprocessor according to clause 6,
   wherein the controller generates an alarm signal in a case of performing the control in the second mode for longer than a third predetermined time period.
Clause 8: The endoscope reprocessor according to clause 2,
   wherein, before switching to the second mode, the controller controls the fluid supplying unit in a fourth mode in which a gas is supplied to the first tube for a predetermined time period.
Clause 9: The endoscope reprocessor according to clause 1, wherein
   the fluid supplying unit includes a pump configured to supply the fluid, and a solenoid valve, and
   the controller controls at least one of the pump or the solenoid valve, to thereby perform the switching between the modes. p1 Clause 10: The endoscope reprocessor according to clause 2,
   wherein the fluid is a liquid, or a gas-liquid mixed flow to alternately supply the liquid or a gas.
Clause 11: The endoscope reprocessor according to clause 2, wherein
   the fluid supplying unit includes a plurality of pumps configured to supply the fluid, and
   the controller controls, in the second mode, the fluid supplying unit so as to supply the fluid by using a larger number of pumps than a number of pumps in the first mode.
Clause 12: The endoscope reprocessor according to clause 2, wherein
   the plurality of thresholds include a third threshold for detecting a connection between the first connector and the first ferrule, and
   based on a result of a comparison between the pressure and the third threshold, the controller generates an alarm signal.
Clause 13: The endoscope reprocessor according to clause 2, wherein
   the plurality of thresholds include a first threshold for detecting contaminants adhering to the conduit and a second threshold for detecting a clogging of the gap due to the contaminants, and
   when the result of the comparison indicates that the flow rate is equal to or greater than the first threshold and lower than the second threshold, the controller switches the control of the fluid supplying unit to the second mode.
Clause 14: An operation method for an endoscope reprocessor for an endoscope,
   the endoscope including: a first conduit communicating with a first ferrule; a second conduit communicating with a second ferrule; and a third conduit in which the first conduit and the second conduit are merged, the third conduit including an opening at a distal end of an insertion portion of the endoscope,
   the operation method comprising:
      controlling a fluid supplying unit in a first mode in which a fluid at a first pressure is supplied to the first conduit;
      acquiring, from a sensor configured to detect a pressure or a flow rate of a fluid supplied to a conduit of the endoscope, the pressure or the flow rate; and
      switching, based on the pressure or the flow rate, the control of the fluid supplying unit from the first mode to a second mode in which the fluid at a second pressure higher than the first pressure is supplied to the first conduit.
Clause 15: The operation method for the endoscope reprocessor for the endoscope according to clause 14, wherein
   the endoscope is connected to a first connector of the endoscope reprocessor at the first ferrule,
   the first connector is configured such that a part of the fluid supplied from a side of the first connector leaks out from a gap between the first connector and the first ferrule to an outer circumferential surface of the first ferrule, and
   the operation method includes comparing the pressure or the flow rate with a plurality of thresholds corresponding to a model of the endoscope acquired in advance, and based on a result of the comparison, switching the control of the fluid supplying unit from the first mode to a second mode, or to a third mode in which the fluid is supplied to the first conduit and the second conduit.
Clause 16: The operation method for the endoscope reprocessor for the endoscope according to clause 15, wherein
   the plurality of thresholds include a first threshold for detecting contaminants adhering to the conduit and a second threshold for detecting a clogging of the gap due to the contaminants, and
   the operation method includes switching the control of the fluid supplying unit to the second mode when the result of the comparison indicates that the pressure is equal to or lower than the first threshold and greater than the second threshold.
Clause 17: The operation method for the endoscope reprocessor for the endoscope according to clause 16, wherein
   the operation method includes switching the control of the fluid supplying unit to the third mode, after a first predetermined time period has elapsed from the switching of the control of the fluid supplying unit to the second mode, or when the pressure becomes equal to or lower than the second threshold.
Clause 18. A control apparatus comprising,
   a processor,
   the processor being configured to:
      control a fluid supplying unit in a first mode in which a fluid at a first pressure is supplied to a first conduit;
      acquire, from a sensor configured to detect a pressure or a flow rate of a fluid supplied to a conduit of an endoscope, the pressure or the flow rate, the endoscope including a first conduit communicating with a first ferrule, a second conduit communicating with a second ferrule, and a third conduit in which the first conduit and the second conduit are merged, the third conduit including an opening at a distal end of an insertion portion of the endoscope; and
      switch, based on the pressure or the flow rate, the control of the fluid supplying unit from the first mode to a second mode in which the fluid at a second pressure higher than the first pressure is supplied to the first conduit.
Clause 19: The control apparatus according to clause 18, wherein
   the endoscope is connected to a first connector at the first ferrule;
   the first connector is configured such that a part of the fluid supplied from a side of the first connector leaks out from a gap between the first connector and the first ferrule to an outer circumferential surface of the first ferrule, and
   the processor compares the pressure or the flow rate with a plurality of thresholds corresponding to a model of the endoscope acquired in advance, and
   switches, based on a result of the comparison, the control of the fluid supplying unit from the first mode to the second mode, or to a third mode in which the fluid is supplied to the first conduit and the second conduit.
Clause 20: The control apparatus according to clause 19, wherein
   the plurality of thresholds include a first threshold for detecting contaminants adhering to the conduit and a second threshold for detecting a clogging of the gap due to the contaminants, and
   when the result of the comparison indicates that the pressure is equal to or lower than the first threshold and greater than the second threshold, the processor switches the control of the fluid supplying unit to the second mode.
Clause 21: The control apparatus according to clause 20,
   wherein the processor switches the control of the fluid supplying unit to the third mode, after a first predetermined time period has elapsed from the switching of the control of the fluid supplying unit to the second mode, or when the pressure becomes equal to or lower than the second threshold.

What is claimed is:

1. An endoscope reprocessor comprising:
a first tube connectable to a first ferrule of an endoscope;
a second tube connectable to a second ferrule of the endoscope;
a fluid supplying assembly configured to supply a fluid to a conduit of the endoscope via the first and second tubes;
a sensor configured to detect at least one of a pressure or a flow rate of the fluid in the conduit; and
processing circuitry configured to:
control the fluid supplying assembly in a first mode to supply the fluid at a first pressure to the first tube;
acquire at least one of the pressure or the flow rate from the sensor; and
based on at least one of the pressure or the flow rate, switch control of the fluid supplying assembly from the first mode to a second mode to supply the fluid at a second pressure higher than the first pressure to the first tube.

2. The endoscope reprocessor according to claim 1, wherein
the first tube includes a first connector configured to be connected to the first ferrule of the endoscope and to allow the fluid supplied from the first tube to partially leak out from a gap between the first connector and the first ferrule to an outer circumferential surface of the first ferrule, and
the processing circuitry is further configured to:
compare at least one of the pressure or the flow rate with a plurality of thresholds corresponding to a model of the endoscope; and
based on a result of the comparison, switch the control of the fluid supplying assembly from the first mode to either the second mode or a third mode in which the fluid is supplied to the first tube and the second tube.

3. The endoscope reprocessor according to claim 2, wherein
the plurality of thresholds include a first threshold for detecting contaminants adhering to the conduit and a second threshold for detecting a clogging of the gap due to the contaminants, and
the processing circuitry is further configured to switch the control of the fluid supplying assembly to the second mode when the result of the comparison indicates that the pressure is equal to or lower than the first threshold and greater than the second threshold.

4. The endoscope reprocessor according to claim 3,
wherein the processing circuitry is further configured to switch the control of the fluid supplying assembly to the third mode, when at least one of a first condition in which a predetermined time period has elapsed from when the control of the fluid supplying assembly was switched to the second mode, or a second condition in which the pressure is equal to or lower than the second threshold, is satisfied.

5. The endoscope reprocessor according to claim 4,
wherein the processing circuitry is further configured to switch the control of the fluid supplying assembly to either a mode in which the processing circuitry starts controlling the fluid supplying assembly from the first mode or a mode in which the processing circuitry starts controlling the fluid supplying assembly from the third mode, according to the model of the endoscope.

6. The endoscope reprocessor according to claim 2,
wherein the processing circuitry is further configured to switch the control of the fluid supplying assembly to the first mode again, after a first particular time period has elapsed from when the control of the fluid supplying assembly was switched to the second mode.

7. The endoscope reprocessor according to claim 6,
wherein the processing circuitry is further configured to generate an alarm signal when the control of the fluid supplying assembly in the second mode is performed for longer than a second particular time period.

8. The endoscope reprocessor according to claim 2,
wherein the processing circuitry is further configured to control, before switching the control of the fluid supplying assembly to the second mode, the fluid supplying assembly in a fourth mode to supply a gas to the first tube for a specific time period.

9. The endoscope reprocessor according to claim 1, wherein
the fluid supplying assembly includes a pump configured to supply the fluid, and a solenoid valve, and
the processing circuitry is further configured to control at least one of the pump or the solenoid valve, thereby switching the control of the fluid supplying assembly between different modes.

10. The endoscope reprocessor according to claim 2,
wherein the fluid supplying assembly is further configured to supply, as the fluid, either a liquid or a gas-liquid mixed flow in which the liquid and a gas are alternately supplied.

11. The endoscope reprocessor according to claim 2, wherein
the fluid supplying assembly includes a plurality of pumps configured to supply the fluid, and
the processing circuitry is further configured to control the fluid supplying assembly to supply the fluid using a larger number of pumps in the second mode than in the first mode.

12. The endoscope reprocessor according to claim 2, wherein
the plurality of thresholds include a particular threshold for detecting a connection between the first connector and the first ferrule, and
the processing circuitry is further configured to generate an alarm signal based on a result of a comparison between the pressure and the particular threshold.

13. The endoscope reprocessor according to claim 2, wherein
the plurality of thresholds include a first threshold for detecting contaminants adhering to the conduit and a second threshold for detecting a clogging of the gap due to the contaminants, and
the processing circuitry is further configured to switch the control of the fluid supplying assembly to the second mode when the result of the comparison indicates that the flow rate is equal to or greater than the first threshold and lower than the second threshold.

14. A method implementable by processing circuitry operatively coupled to an endoscope reprocessor, the method comprising:
controlling a fluid supplying assembly in a first mode to supply a fluid at a first pressure to a first tube;
acquiring at least one of a pressure or a flow rate of the fluid in a conduit of an endoscope from a sensor; and
based on at least one of the pressure or the flow rate, switching control of the fluid supplying assembly from the first mode to a second mode to supply the fluid at a second pressure higher than the first pressure to the first tube,
wherein the endoscope reprocessor comprises:
the first tube connectable to a first ferrule of the endoscope;
a second tube connectable to a second ferrule of the endoscope;
the fluid supplying assembly configured to supply the fluid to the conduit of the endoscope via the first and second tubes; and
the sensor configured to detect at least one of the pressure or the flow rate of the fluid in the conduit.

15. The method according to claim 14, wherein
the first tube includes a first connector configured to be connected to the first ferrule of the endoscope and to allow the fluid supplied from the first tube to partially leak out from a gap between the first connector and the first ferrule to an outer circumferential surface of the first ferrule, and
the switching includes:
comparing at least one of the pressure or the flow rate with a plurality of thresholds corresponding to a model of the endoscope; and
based on a result of the comparison, switching the control of the fluid supplying assembly from the first mode to either the second mode or a third mode in which the fluid is supplied to the first tube and the second tube.

16. The method according to claim 15, wherein
the plurality of thresholds include a first threshold for detecting contaminants adhering to the conduit and a second threshold for detecting a clogging of the gap due to the contaminants, and
the switching further includes switching the control of the fluid supplying assembly to the second mode when the result of the comparison indicates that the pressure is equal to or lower than the first threshold and greater than the second threshold.

17. The method according to claim 16,
wherein the switching further includes switching the control of the fluid supplying assembly to the third mode when at least one of a first condition in which a predetermined time period has elapsed from when the control of the fluid supplying assembly was switched to the second mode, or a second condition in which the pressure is equal to or lower than the second threshold, is satisfied.

18. A control apparatus comprising,
a connection interface connectable to:
a fluid supplying assembly configured to supply a fluid to a conduit of an endoscope via a first tube and a second tube, the first tube being connectable to a first ferrule of the endoscope, the second tube being connectable to a second ferrule of the endoscope; and
a sensor configured to detect at least one of a pressure or a flow rate of the fluid in the conduit; and
processing circuitry configured to:
control the fluid supplying assembly in a first mode to supply the fluid at a first pressure to the first tube;
acquire at least one of the pressure or the flow rate from the sensor; and
based on at least one of the pressure or the flow rate, switch control of the fluid supplying assembly from the first mode to a second mode to supply the fluid at a second pressure higher than the first pressure to the first tube.

19. The control apparatus according to claim 18, wherein
the first tube includes a first connector configured to be connected to the first ferrule of the endoscope and to allow the fluid supplied from the first tube to partially leak out from a gap between the first connector and the first ferrule to an outer circumferential surface of the first ferrule, and
the processing circuitry is further configured to:
compare at least one of the pressure or the flow rate with a plurality of thresholds corresponding to a model of the endoscope; and
based on a result of the comparison, switch the control of the fluid supplying assembly from the first mode to either the second mode or a third mode in which the fluid is supplied to the first tube and the second tube.

20. The control apparatus according to claim 19, wherein
the plurality of thresholds include a first threshold for detecting contaminants adhering to the conduit and a second threshold for detecting a clogging of the gap due to the contaminants, and
the processing circuitry is further configured to switch the control of the fluid supplying assembly to the second mode when the result of the comparison indicates that the pressure is equal to or lower than the first threshold and greater than the second threshold.

21. The control apparatus according to claim 20,
wherein the processing circuitry is further configured to switch the control of the fluid supplying assembly to the third mode, when at least one of a first condition in which a predetermined time period has elapsed from when the control of the fluid supplying assembly was switched to the second mode, or a second condition in which the pressure is equal to or lower than the second threshold, is satisfied.

22. The control apparatus according to claim 18,
wherein the processing circuitry comprises:
a processor; and
a non-transitory computer-readable storage medium storing computer-readable instructions configured to, when executed by the processor, cause the processor to:
control the fluid supplying assembly in the first mode to supply the fluid at the first pressure to the first tube;
acquire at least one of the pressure or the flow rate from the sensor; and
based on at least one of the pressure or the flow rate, switch the control of the fluid supplying assembly from the first mode to the second mode to supply the fluid at the second pressure to the first tube.
